(19) United States
(12) Patent Application Publication
(10) Pub. No.: US 2005/0123958 A1
(43) Pub. Date: Jun. 9, 2005

(54) Method of removing mismatch bound polynucleotides

(75) Inventor: Toru Tsuchiya, Kanagawa-ken (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 10/953,445

(22) Filed: Sep. 30, 2004

(30) Foreign Application Priority Data
Sep. 30, 2003 (JP) (PAT)339714/2003

(51) Int. Cl. C12Q 1/68; C12P 19/34

(52) U.S. Cl. 435/6; 435/91.2

(57) ABSTRACT

A labeled polynucleotide is subjected to hybridization with oligonucleotide probes fixed respectively to regions on a supporting material. With a restriction enzyme, a single-stranded moiety of the labeled polynucleotide having failed to form a double strand with each probe is separated from a double strand having been formed by the labeled polynucleotide and each probe. Thereafter, an electrode is located at a surface of each region on the supporting material, and a voltage is applied to the electrode. The labeled polynucleotide, which has undergone a mismatch binding with a certain oligonucleotide probe, is thus separated from the corresponding region on the supporting material.

### BACKGROUND OF THE INVENTION

1. Field of the Invention

This invention relates to a method of removing a mismatch bound polynucleotide from a biochemical analysis micro array used for detection, analysis, or the like, of a specific sequence contained in a polynucleotide, such as a DNA.

2. Description of the Related Art

DNA micro arrays are expected to be applied to a wide range of fields of life science, such as monitoring of genetic expression, determination of base sequences of genes, analysis of gene polymorphism (SNP), analysis of gene amplification or deletion at cancered parts, classification of diseases, such as cancers, prediction of drug response characteristics, and searching of disease genes.

Principles of assay techniques utilizing DNA micro arrays are based upon detection of nucleic acids through hybridization. Specifically, various different probe DNA's are arrayed at a high density at a plurality of regions of a surface of a supporting material, such as glass, silicon, or a membrane filter, and secured to the regions of the surface of the supporting material. Thereafter, a target DNA (i.e., a DNA having been labeled with a labeling substance) is subjected to hybridization with the probe DNA's having been fixed to the regions of the surface of the supporting material. Signals obtained from the regions (spots) are then detected in the manner described below.

For example, in cases where the target DNA has been labeled with a radioactive labeling substance, a stimulable phosphor layer of a stimulable phosphor sheet is exposed to radiation radiated out from the radioactive labeling substance, which is contained selectively in the regions of the supporting material. Thereafter, the stimulable phosphor layer is exposed to stimulating rays, which cause the stimulable phosphor layer to emit light in proportion to the amount of energy stored on the stimulable phosphor layer during the exposure of the stimulable phosphor layer to the radiation. The light emitted by the stimulable phosphor layer is detected photoelectrically. In this manner, the target DNA having been specifically bound to at least one of the probe DNA's, which have been fixed to the regions of the surface of the supporting material, is detected.

In cases where the target DNA has been labeled with a fluorescent labeling substance, excitation light is irradiated to the regions of the supporting material, and the fluorescent labeling substance, which is contained selectively in the regions of the supporting material, is excited by the excitation light to produce fluorescence. The thus produced fluorescence is detected photoelectrically.

In cases where the target DNA has been labeled with a chemical luminescent labeling substance capable of producing the chemical luminescence when being brought into contact with a chemical luminescence substrate, the chemical luminescent labeling substance, which is contained selectively in the regions of the supporting material, is brought into contact with the chemical luminescence substrate. Also, the chemical luminescence produced by the chemical luminescent labeling substance is detected photoelectrically. (The aforesaid assay techniques are described in, for example, U.S. Patent Laid-Open No. 20020016009.)

The DNA micro arrays may be classified into two groups in accordance with the kinds of the DNA's, which are arrayed, and processes for producing the DNA micro arrays. One of the two groups is an oligonucleotide array produced with a process, wherein a light blocking plate referred to as a mask is overlaid on a silicon base plate, the silicon base plate is exposed to light via the mask by the utilization of photo-lithography, which is an exposure technique for semiconductors, the operation for exposing the silicon base plate to light via the mask is iterated, and DNA molecules are thereby superposed one by one on the base plate. (The oligonucleotide array produced with the process described above will hereinbelow be referred to simply as the oligonucleotide array.) The other group is a cDNA micro array produced with a process, wherein cDNA's having been subjected to PCR amplification previously are spotted onto slide glass by use of a thin pin, an ink jet technique, or the like.

Conditions (such as a temperature and a salt concentration) optimum for the hybridization may vary for the different kinds of the probe DNA's having been fixed respectively to the regions. However, it is not always possible to perform the reaction under the conditions optimum for each of the probe DNA's, which have been fixed respectively to the regions having been located at a high density. Therefore, ordinarily, the reaction is performed under the identical conditions with respect to all of the regions. Accordingly, it may often occur that a target DNA, which is not perfectly complementary to a certain probe DNA on the array and has a sequence similar to the perfectly complementary sequence, undergoes incorrect hybridization with the aforesaid certain probe DNA on the array. The incorrect hybridization described above is referred to as the mismatch binding. The target DNA, which has undergone the mismatch binding, causes noise to occur at the time of signal detection and adversely affects a detection accuracy.

In particular, the cDNA micro array is produced by directly subjecting the cDNA's, which have been isolated from cells of organisms, to the PCR amplification and fixed to the slide glass. The cDNA micro array is not produced by previously designing the probe DNA's so as not to undergo a mismatch bonding as in the cases of the oligonucleotide array. Therefore, the cDNA micro array has a high possibility that the probe DNA's on the cDNA micro array will undergo the mismatch binding. Accordingly, in the cases of the cDNA micro array, the mismatch binding is suppressed through preparation of the probes located on the cDNA micro array. For such purposes, for example, sequences specific to a gene to be detected are selected, and oligo DNA's having been synthesized in accordance with the selected sequences are used as the probes.

However, in both the cases of the oligonucleotide array and the synthetic oligo array, it is not always possible to design such that the mismatch binding does not occur. Particularly, in cases where analysis is to be made with respect to a long gene, such as a cDNA, it is almost impossible to design such that the mismatch binding does not occur.

Attempts have been made to solve the problems with regard to the mismatch binding described above by, for example, finely adjusting the temperature or the pH value at the time of hybridization of a target DNA with probe DNA's having been fixed to an array. For example, a biochemical reaction detecting chip for the hybridization of a polynucleotide with oligonucleotide probes, with which biochemical reaction detecting chip the biochemical reaction is capable of being caused to occur at a temperature optimum for the hybridization at each of probe fixing surfaces, has been proposed in, for example, U.S. Patent Laid-Open No. 20020164778. The proposed biochemical reaction detecting chip is based upon characteristics concerning a melting out temperature (i.e., a Tm value) of a complementary strand binding of oligonucleotide probes. Specifically, under conditions of temperatures lower than the Tm value, background noise due to the mismatch binding increases. Also, under conditions of temperatures higher than the Tm value, it becomes difficult for the polynucleotide to undergo the binding with the probes. Therefore, with the proposed biochemical reaction detecting chip, a temperature, at which the polynucleotide is capable of undergoing the hybridization with a probe such that the mismatch binding does not occur, is adjusted at a value optimum for each of the probes.

Also, a technique for selectively separating and recovering a desired polynucleotide is proposed in, for example, U.S. Pat. No. 6,093,370. With the proposed technique for selectively separating and recovering a desired polynucleotide, oligonucleotide probes are fixed respectively to regions of a surface of a base plate, and polynucleotides are subjected to the hybridization with the oligonucleotide probes. Thereafter, only a specific region of the base plate is heated selectively, and only the polynucleotide, which has been complementarily bound to the probe, is separated from the probe.

However, with the technique for utilizing the biochemical reaction detecting chip proposed in U.S. Patent Laid-Open No. 20020164778, it is necessary that a plurality of islands are formed on the biochemical reaction detecting chip, and that the temperature adjustment is monitored finely for each of the islands. Also, in cases where the technique for selectively separating and recovering a desired polynucleotide, which is proposed in U.S. Pat. No. 6,093,370, is utilized, it is necessary that only the specific region of the base plate is heated selectively. Further, the Tm values of the probes, which are contained in the islands or the regions described above, must be set previously at approximately identical values. As described above, with each of the technique for utilizing the biochemical reaction detecting chip proposed in U.S. Patent Laid-Open No. 20020164778 and the technique for selectively separating and recovering a desired polynucleotide, which is proposed in U.S. Pat. No. 6,093,370, complicated operations must be performed, and it is necessary for a specific apparatus to be utilized. Furthermore, in order for the mismatch binding to be suppressed, fine adjustment for raising or lowering the salt concentration in a liquid subjected to reaction must be made, and considerable labor and time are thus required. In cases where the hybridization is performed through adjustment of the pH value, the same problems as those described above arise.

As described above, in order for the mismatch binding during the hybridization to be suppressed, complicated adjustments must be performed, and considerable labor and time are required. Also, new problems occur in that, depending upon the conditions for the suppression of the mismatch binding, perfect match binding, i.e. perfect complementary binding, is weakened.

With conventional assay techniques, from the view point of removing a target DNA having undergone a mismatch binding, after a hybridization liquid containing a target DNA has been subjected to reaction with an array having regions, to which probe DNA's have respectively been fixed, a liquid washing operation utilizing a washing liquid is performed in order to remove surplus target DNA remaining in the regions of the array.

However, the target DNA, which has undergone the mismatch binding with the probe DNA's having been fixed to the regions of the array, has been bound to the probe DNA's partially or by a certain kind of interaction. Therefore, with the conventional washing technique utilizing the liquid washing, it is not always possible to achieve uniform control of the washing intensity, and accurate washing is not capable of being performed.

A technique for using a unit, in which a gene has been fitted onto an electrode, is disclosed in, for example, U.S. Pat. No. 5,605,662. The disclosed technique aims at removing a mismatch binding by the utilization the characteristics in that the gene is charged negatively.

However, with the disclosed technique for using a unit, in which a gene has been fitted onto an electrode, in cases where the target DNA, which has been bound to each of the probe DNA's, has one of various different lengths, physical force applied to the target DNA at the time of application of a voltage to the electrode varies for different target DNA lengths. Therefore, it is presumed that both the target DNA, which has undergone the mismatch binding with the probe DNA's, and the target DNA, which has undergone the perfect match binding with the probe DNA's, are separated from the probe DNA's.

### SUMMARY OF THE INVENTION

The primary object of the present invention is to provide a method of removing a mismatch bound polynucleotide, wherein only a mismatch bound polynucleotide, such as a target DNA, which has undergone a mismatch binding, is capable of being removed, while a perfect match bound polynucleotide is being kept unremoved.

The present invention provides a method of removing a mismatch bound polynucleotide, comprising the steps of:

i) subjecting a labeled polynucleotide, which has been labeled with a labeling substance, to hybridization with a plurality of oligonucleotide probes, which have been fixed respectively to a plurality of regions on a supporting material,

ii) causing a restriction enzyme to act upon a single-stranded moiety of the labeled polynucleotide, which moiety has failed to form a double strand with each of the plurality of the oligonucleotide probes having been respectively fixed to the plurality of the regions on the supporting material, the restriction enzyme being capable of decomposing a single-stranded polynucleotide from a terminal of the labeled polynucleotide, the single-stranded moiety of the labeled polynucleotide being thereby separated from a double strand, which has been formed by the labeled polynucleotide and each of the oligonucleotide probes,

iii) locating at least one electrode such that the electrode is capable of applying a voltage across each of the regions on the supporting material after the single-stranded moiety of the labeled polynucleotide has been separated from the double strand, which has been formed by the labeled polynucleotide and each of the oligonucleotide probes, and

iv) applying a voltage to the electrode, whereby the labeled polynucleotide, which has undergone a mismatch binding with a certain oligonucleotide probe among the plurality of the oligonucleotide probes having been respectively fixed to the plurality of the regions on the supporting material, is separated from a region on the supporting material, to which region the labeled polynucleotide having undergone the mismatch binding has been bound.

In the method of removing a mismatch bound polynucleotide in accordance with the present invention, such that identical physical force may be exerted by the applied voltage and upon the labeled polynucleotide at each of the regions on the supporting material, the oligonucleotide probes are formed from nucleotides, which have approximately identical lengths, with respect to all of the regions on the supporting material. Also, the applied voltage is set such that the labeled polynucleotide, which has undergone a perfect match binding with each of the oligonucleotide probes, may not be separated from the corresponding region on the supporting material, and such that only the labeled polynucleotide, which has undergone the mismatch binding with the certain oligonucleotide probe, may be separated from the corresponding region on the supporting material.

The method of removing a mismatch bound polynucleotide in accordance with the present invention should preferably be modified such that the restriction enzyme is exonuclease VII.

With the method of removing a mismatch bound polynucleotide in accordance with the present invention, after the labeled polynucleotide, which has been labeled with the labeling substance, has been subjected to the hybridization with the plurality of the oligonucleotide probes, which have been fixed respectively to the plurality of the regions on the supporting material, the restriction enzyme is caused to act upon the single-stranded moiety of the labeled polynucleotide, which moiety has failed to form the double strand with each of the plurality of the oligonucleotide probes having been respectively fixed to the plurality of the regions on the supporting material. The restriction enzyme is capable of decomposing the single-stranded polynucleotide from the terminal of the labeled polynucleotide. The single-stranded moiety of the labeled polynucleotide is thereby separated from the double strand, which has been formed by the labeled polynucleotide and each of the oligonucleotide probes. A nucleotide base length of the labeled polynucleotide is thus capable of being set to be identical at each of the regions on the supporting material. Thereafter, the at least one electrode is located such that the electrode is capable of applying the voltage across each of the regions on the supporting material, and the voltage is applied to the electrode. Therefore, with the method of removing a mismatch bound polynucleotide in accordance with the present invention, only the labeled polynucleotide, which has undergone the mismatch binding with the certain oligonucleotide probe, is capable of being separated from the corresponding region on the supporting material, while the labeled polynucleotide, which has undergone the perfect match binding with each of the oligonucleotide probes, is being kept unseparated from the corresponding region on the supporting material. Accordingly, background noise due to the mismatch binding is capable of being suppressed, and the detection accuracy is capable of being enhanced.

### BRIEF DESCRIPTION OF THE DRAWINGS

FIGS. 1A to 1E are explanatory views showing how a mismatch bound DNA is removed with an embodiment of the method of removing a mismatch bound polynucleotide in accordance with the present invention, and

FIG. 2 is a schematic sectional view showing an example of a cleaning apparatus, in which a plurality of electrodes are located.

### DESCRIPTION OF THE PREFERRED EMBODIMENTS

The present invention will hereinbelow be described in further detail with reference to the accompanying drawings.

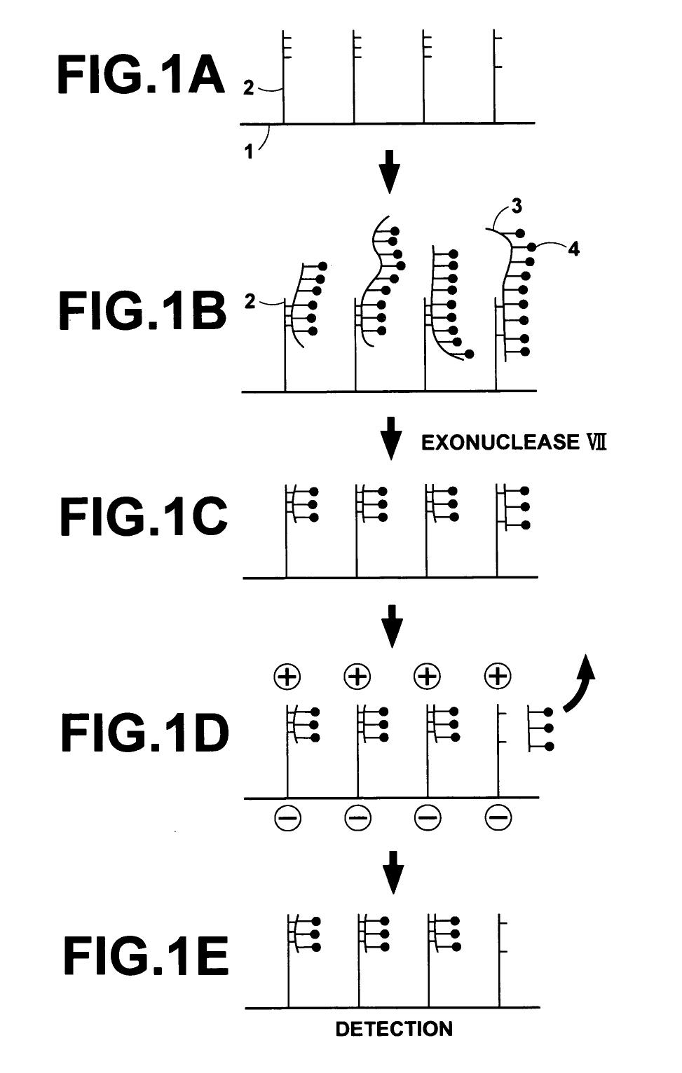

An embodiment of the method of removing a mismatch bound polynucleotide in accordance with the present invention, wherein a DNA array comprising a supporting material and a plurality of oligo DNA probes having been fixed respectively to a plurality of regions on the supporting material is utilized, will be described hereinbelow. FIGS. 1A to 1E are explanatory views showing how a mismatch bound DNA is removed with an embodiment of the method of removing a mismatch bound polynucleotide in accordance with the present invention.

Firstly, a surface of a membrane filter 1 acting as a supporting material is processed such that carboxyl groups (COOH) or aldehyde groups (COH) are exposed from the surface of the membrane filter 1 to the exterior. Also, an amino group (NH2) is introduced into a 5′-terminal of each of synthetic oligo DNA's acting as DNA probes 2, 2, . . . Each of the DNA probes 2, 2, . . . having the terminals, to which the amino groups have been introduced, is spotted onto the membrane filter 1, which has been subjected to the surface processing. As a result, a covalent bond is formed between the carboxyl group or the aldehyde group, which is exposed on the surface of the membrane filter 1, and the amino group, which has been introduced into the terminal of each of the DNA probes 2, 2, . . . In this manner, as illustrated in FIG. 1A, the DNA probes 2, 2, . . . are fixed to the membrane filter 1.

In FIG. 1A, part of each of the DNA probes 2, 2, . . . , which part extends vertically from the membrane filter 1, represents a sequence of the DNA. Also, each of parts, which branch out from the vertically extending part of each of the DNA probes 2, 2, . . . , represents the part, which complementarily forms a base pair. The DNA probes 2, 2, . . . having different sequences are respectively spotted onto regions of the membrane filter 1. The DNA probes 2, 2, . . . are prepared such that the DNA lengths are approximately identical with one another at all of the regions of the membrane filter 1. In FIG. 1A, as an aid in facilitating the explanation, one DNA probe 2 is fixed to one region (i.e., one spot) on the membrane filter 1. In this manner, only one DNA probe 2 may be fixed to each of the regions of the membrane filter 1. Alternatively, several DNA probes may be fixed to each of the regions of the membrane filter 1. Also, the DNA probes 2, 2, . . . may be the synthesized oligo DNA's. Alternatively, the DNA probes 2, 2, . . . may be the cDNA's having been isolated from cells of organisms.

Thereafter, a labeled DNA acting as a target is prepared. As the DNA of the labeled DNA, a total RNA, a poly-A+RNA, or the like, which has been extracted from a cell or a tissue of an organism and purified, is subjected to reverse transcription using a reverse transcriptase, and a cDNA is thereby prepared. The labeled DNA may be prepared by incorporating a labeling substance into the cDNA during the reverse transcription using the reverse transcriptase. Also, biotin or DNP (dinitrophenyl) may be incorporated into the cDNA during the reverse transcription, such that a signal may be amplified via an antibody reaction or an enzyme reaction.

The labeling substance may be selected from various labeling substances, for which the regularity of incorporation into the cDNA is known previously. By way of example, the labeling substance may be a fluoro chrome, such as Cy3, Cy5, or fluorescein isothiocyanate. Alternatively, the labeling substance may be a radioactive isotope, such as 32P or 33P. As another alternative, the labeling substance maybe a labeling substance for chemical luminescence, such as alkaline phosphatase, peroxidase, luciferase, biotin, or digoxigenin.

Thereafter, a labeled DNA 3 is subjected to hybridization reaction with the DNA probes 2, 2, . . . , which have been fixed to the membrane filter 1. By way of example, the hybridization reaction may be performed with a process, wherein the membrane filter 1, to which the DNA probes 2, 2, . . . have been fixed, and a reaction liquid containing the labeled DNA 3 are put into a hybridization bag, vibrations are given to the hybridization bag, and the labeled DNA 3 is thereby moved through convection or diffusion in the hybridization bag. Alternatively, the hybridization reaction may be performed with a process utilizing a reactor provided with a pump, a syringe, or the like, in which the reaction liquid containing the labeled DNA 3 is capable of being forcibly caused to flow across each of the regions of the membrane filter 1.

After the hybridization reaction has been performed, such that the surplus labeled DNA, which has not been bound to the DNA probes 2, 2, . . . through the hybridization, may be removed, a washing liquid may be introduced into the hybridization bag or the reactor in order to wash the membrane filter 1.

As illustrated in FIG. 1B, after the hybridization reaction has been performed, the labeled DNA 3, which has been labeled with a labeling substance 4 and has one of various different lengths, complementarily forms the base pair through the hydrogen bond with each of the DNA probes 2, 2, . . . (The DNA probe 2 and the labeled DNA 3 illustrated on the right end side of FIG. 1B are in a state in which they have moieties having not undergone a perfect complementary binding.) If application of a voltage across each of the regions on the membrane filter 1 is performed in the state in which the labeled DNA 3 has one of various different lengths in the manner described above, the physical force exerted upon the labeled DNA 3 acting as the target will vary in accordance with the variance in length. Therefore, in such cases, the problems will occur in that both the labeled DNA 3, which has undergone the mismatch binding with a DNA probe 2, and the labeled DNA 3, which has undergone the perfect complementary binding, i.e. the perfect match binding, with a DNA probe 2, are separated from the corresponding DNA probes 2, 2, and the selective separation of only the labeled DNA 3, which has undergone the mismatch binding with the DNA probe 2, is not capable of being performed. Therefore, in this embodiment, by use of exonuclease VII, a single-stranded moiety of the labeled DNA 3, which moiety has failed to form a double strand with each of the DNA probes 2, 2, . . . , is separated from the double strand, which has been formed by the labeled DNA 3 and each of the DNA probes 2, 2, . . . Exonuclease VII is a restriction enzyme capable of specifically acting upon a terminal of a single-stranded DNA and decomposing the single-stranded polynucleotide. In cases where the labeled DNA 3 having been bound to each of the DNA probes 2, 2, . . . is processed with exonuclease VII at a temperature falling within the range of 5° C. to 20° C., as illustrated in FIG. 1C, the single-stranded moiety of the labeled DNA 3, which moiety has failed to form a double strand with each of the DNA probes 2, 2, . . . , is separated from the double strand, which has been formed by the labeled DNA 3 and each of the DNA probes 2, 2, . . . , and only the moieties, at which the labeled DNA 3 and each of the DNA probes 2, 2, . . . have formed the double strands, remain at each of the regions of the membrane filter 1.

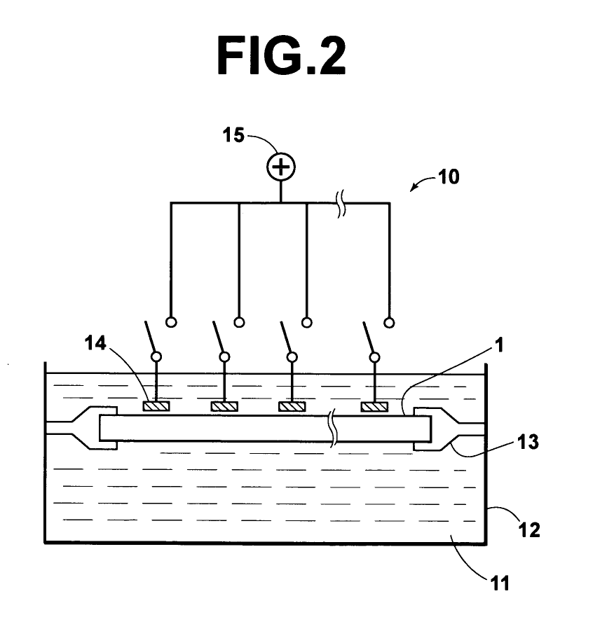

In the state illustrated in FIG. 1C, electrodes are located such that a voltage is capable of being applied across each of the regions of the membrane filter 1. Also, as illustrated in FIG. 1D, the voltage is applied across each of the regions of the membrane filter 1. An example of a cleaning apparatus, in which the electrodes are located such that a voltage is capable of being applied across each of the regions of the membrane filter 1, will be described hereinbelow. FIG. 2 is a schematic sectional-view showing an example of a cleaning apparatus, in which a plurality of electrodes are located. As illustrated in FIG. 2, the cleaning apparatus comprises a cleaning vessel 12 for accommodating a cleaning liquid 11 therein and an electric field forming device 10 acting as control means. A membrane filter support section 13 capable of supporting the membrane filter 1 is formed within the cleaning vessel 12. The electric field forming device 10 is constituted of a plurality of electrodes 14, 14, . . . and a positive electric power source 15. Each of the electrodes 14, 14, . . . is located at a surface corresponding to one of the regions of the membrane filter 1.

After the cleaning liquid 11 has been accommodated within the cleaning vessel 12, each of the electrodes 14, 14, . . . is electrically connected to the positive electric power source 15. As a result, a positive voltage is applied to each of the electrodes 14, 14, . . . , and an electric current flows across the region having the surface, at which the electrode 14 has been located. The binding force of the labeled DNA 3, which has undergone the mismatch binding with a DNA probe 2, is smaller than the binding force of the labeled DNA 3, which has undergone the perfect match binding with a DNA probe 2. Therefore, the applied voltage is adjusted such that only the labeled DNA 3, which has undergone the mismatch binding with a DNA probe 2, is capable of being separated from the corresponding region of the membrane filter 1, while the labeled DNA 3, which has undergone the perfect match binding with a DNA probe 2, is being kept unseparated from the corresponding region of the membrane filter 1. In this manner, only the labeled DNA 3, which has undergone the mismatch binding with a DNA probe 2 having been bound to the corresponding region of the membrane filter 1, is capable of being selectively attracted toward the corresponding electrode 14. At the time at which the positive electric power source 15 is turned off, the labeled DNA 3, which has been attracted to the surface of the electrode 14, separates from the surface of the electrode 14 and shifts into the cleaning liquid 11. In this manner, as illustrated in FIG. 1E, the labeled DNA 3, which has undergone the mismatch binding with the DNA probe 2, is removed.

As described above, the applied voltage is adjusted such that only the labeled DNA 3, which has undergone the mismatch binding with a DNA probe 2, is capable of being separated from the corresponding region of the membrane filter 1, while the labeled DNA 3, which has undergone the perfect match binding with a DNA probe 2, is being kept unseparated from the corresponding region of the membrane filter 1. The applied voltage may vary in accordance with the lengths of the DNA probes 2, 2, . . . However, the applied voltage should preferably fall within the range of approximately 1V to approximately 2V. The applied voltage may be a d.c. voltage or an a.c. voltage.

In the state illustrated in FIG. 1E, detection of the labeled DNA 3 is performed. The operation for the detection varies in accordance with the kind of the labeling substance 4 with which the labeled DNA 3 has been labeled. For example, in cases where the labeling substance 4 is a fluoro chrome, excitation light is irradiated to the regions of the membrane filter 1, and the fluorescent labeling substance, which is contained selectively in the regions of the membrane filter 1, is excited by the excitation light to produce fluorescence. The thus produced fluorescence is detected photoelectrically by use of a CCD camera, a laser+a PMT, or the like.

In cases where the labeling substance 4 is a radioactive isotope, a stimulable phosphor layer of a stimulable phosphor sheet is exposed to radiation radiated out from the radioactive labeling substance, which is contained selectively in the regions of the membrane filter 1. Thereafter, the stimulable phosphor layer is exposed to stimulating rays, which cause the stimulable phosphor layer to emit light in proportion to the amount of energy stored on the stimulable phosphor layer during the exposure of the stimulable phosphor layer to the radiation. The light emitted by the stimulable phosphor layer is detected photoelectrically.

In cases where the labeling substance 4 is a chemical luminescent labeling substance, such as an enzyme, which is capable of producing the chemical luminescence when being brought into contact with a chemical luminescence substrate, the chemical luminescent labeling substance, which is contained selectively in the regions of the membrane filter 1, is brought into contact with the chemical luminescence substrate. Also, the chemical luminescence produced by the chemical luminescent labeling substance is detected photoelectrically.

The detection data having thus been obtained with the detection is the data obtained in the state in which the labeled DNA 3 having undergone the mismatch binding has been removed. Therefore, the detection data is free from noise due to the labeled DNA 3 having undergone the mismatch binding.

As described above, with this embodiment, after the labeled DNA 3 has been subjected to the hybridization with the DNA probes 2, 2, . . . , the single-stranded moiety of the labeled DNA 3, which moiety has failed to form the double strand with each of the DNA probes 2, 2, . . . , is separated from the double strand, which has been formed by the labeled DNA 3 and each of the DNA probes 2, 2, . . . by the action of exonuclease VII. The DNA lengths of the labeled DNA 3 are thus set at identical lengths among the regions of the membrane filter 1. Thereafter, the voltage is applied to the electrodes 14, 14, . . . in the cleaning apparatus, and the electric current is caused to flow across each of the regions of the membrane filter 1. Therefore, only the labeled DNA 3, which has undergone the mismatch binding with a DNA probe 2, is capable of being selectively separated from the corresponding region of the membrane filter 1, while the labeled DNA 3, which has undergone the perfect match binding with a DNA probe 2, is being kept unseparated from the corresponding region of the membrane filter 1. Accordingly, background noise due to the mismatch binding is capable of being prevented from occurring.

In the embodiment described above, the DNA probes 2, 2, . . . are employed as the oligonucleotide probes. Also, the labeled DNA 3 is employed as the labeled polynucleotide. However, the method of removing a mismatch bound polynucleotide in accordance with the present invention is not limited to the use of the DNA probes 2, 2, . . . and the labeled DNA 3. For example, RNA's, nucleic acid precursors, or coenzymes may be employed in the method of removing a mismatch bound polynucleotide in accordance with the present invention.

1. A method of removing a mismatch bound polynucleotide, comprising the steps of:
i) subjecting a labeled polynucleotide, which has been labeled with a labeling substance, to hybridization with a plurality of oligonucleotide probes, which have been fixed respectively to a plurality of regions on a supporting material,
ii) causing a restriction enzyme to act upon a single-stranded moiety of the labeled polynucleotide, which moiety has failed to form a double strand with each of the plurality of the oligonucleotide probes having been respectively fixed to the plurality of the regions on the supporting material, the restriction enzyme being capable of decomposing a single-stranded polynucleotide from a terminal of the labeled polynucleotide, the single-stranded moiety of the labeled polynucleotide being thereby separated from a double strand, which has been formed by the labeled polynucleotide and each of the oligonucleotide probes,
iii) locating at least one electrode such that the electrode is capable of applying a voltage across each of the regions on the supporting material after the single-stranded moiety of the labeled polynucleotide has been separated from the double strand, which has been formed by the labeled polynucleotide and each of the oligonucleotide probes, and
iv) applying a voltage to the electrode, whereby the labeled polynucleotide, which has undergone a mismatch binding with a certain oligonucleotide probe among the plurality of the oligonucleotide probes having been respectively fixed to the plurality of the regions on the supporting material, is separated from a region on the supporting material, to which region the labeled polynucleotide having undergone the mismatch binding has been bound.

2. A method as defined in claim 1 wherein the restriction enzyme is exonuclease VII.

3. A method as defined in claim 1 wherein the labeling substance is selected from the group consisting of a radioactive labeling substance, a fluorescent labeling substance, and a chemical luminescent labeling substance capable of producing chemical luminescence when being brought into contact with a chemical luminescence substrate.
